(19) United States
(12) Patent Application Publication
(10) Pub. No.: US 2021/0196986 A1
(43) Pub. Date: Jul. 1, 2021

(54) BEAM DELIVERY PLATFORM AND POSITION SYSTEM

(71) Applicant: MEDICAL BEAM LABORATORIES, LLC, GREENVILLE, SC (US)

(72) Inventor: LASZLO FADGYAS, GREENVILLE, SC (US)

(73) Assignee: MEDICAL BEAM LABORATORIES, LLC, GREEENVILLE, SC (US)

(21) Appl. No.: 17/271,030

(22) PCT Filed: Aug. 26, 2019
(86) PCT No.: PCT/US19/48205

§ 371 Date: Feb. 24, 2021

Related U.S. Application Data
(60) Provisional application No. 62/722,488, filed on Aug. 24, 2018.
(60) Provisional application No. 62/722,506, filed on Aug. 24, 2018.

(51) Int. Cl. A61N 5/10

(52) U.S. Cl. CPC A61N 5/1081; A61N 5/1049; A61N 5/107; A61N 2005/1059; A61N 2005/1094; A61N 2005/1055; A61N 2005/1058; A61N 2005/1061; A61N 2005/1063; A61N 2005/1052

(57) ABSTRACT

A radiation beam delivery apparatus is provided that utilizes a plurality of treatment heat actuators to provide accurate positioning of a treatment head relative to the patient. The apparatus fallows for six degrees of freedom for the treatment heat and further provides for a patient support that can be Independently adjusted along a patient-s x axis, a y-axis, and roll and pitch movements.

### RELATED APPLICATIONS

This application claims the benefit of US provisional application having Ser. No. 62/722,488 filed on Aug. 24, 2018 and US provisional application having Ser. No. 82/722,506 filed on Aug. 24, 2018, both applications being incorporated herein by reference for all purposes.

### FIELD OF THE INVENTION

This invention is directed towards a Beam Delivery Platform (BDP) which can precisely position radiation delivery heads along 6 degrees of freedom (DOF) for highly localized, conformal radiation dose distributions within critical target volume with a sharp dose fail off at the boundary of the indication and the surrounding healthy tissue. The invention further relates to a process of administering radiation dose to a patient utilizing a novel platform along with a patent positioning apparatus and process of using the apparatus. This invention is further directed towards a patient positioning system for treatment with a radiation beam delivery radiosurgery system.

### BACKGROUND OF THE INVENTION

This invention relates to support frames used with the delivery of radiation therapy to patients. Support frames know in the art include:

Linac-based C shape gantries which only offer rotational motion of the treatment head about the Z-axis (isocentric rotation).

Conventional rotational ring shape gantries only provide for isocentric rotating motion but do not provide pitching or radial motion.

Brainlab™ Ring Shape Gantry provides isocentric rotational motion and yaw rotational motion, but does not offer pitching or radial motion.

Cyberknife™ allows motion in 6 DOF, but cannot accomrnodato arc isotope-based treatment head with activity suitable for radiosurgery. Furthermore, it cannot accommodate an on-board IGS or radiation beam stopper and cannot support a radiation safely enclosure.

Zap-X (Zap Surgical Systems) provides a radiation shielding enclosure, but it is not capable of full body treatment due to its design.

This invention relates to patient positioning systems for delivery of targeted radiation therapy. Prior art systems include: Hexapod-based PPSs which are patient positioning systems that utilize hexapods but have a very limited movement envelope, especially on patient X axis and Z axis. Such systems are typically used as a fine-tune subassembly add-on feature to existing systems.

SCARA-based PPSs are patient positioning systems which utilize SCARA robotics and require a relatively large working area (envelope). Such systems are not ideal for dynamic motion compensation applications, which may be required for treatments of mobile tumors.

Traditional PPSs allow for motion in only the basic X, Y, and Z linear axes. While some also sit on top of a rotating platform, they are incapable of providing the 6-axis motion required for advanced patent positioning applications.

Accordingly, there remains room for improvement and variation within the art.

### SUMMARY OF THE INVENTION

It is one aspect of at least one of the present embodiments to provide a radiation delivery platform that positions radiation delivery heads for highly localized, conformal radiation dose distributions within clinical target volume with a sharp dose fall off at the boundary of the indication and the surrounding healthy tissue. The BDP can accommodate and hold single or multipleradiation therapy treatment heads including, isotope-based treatment heads like the PRTH, linear accelerator (LINAC) based treatment heads, and particle beam treatment heads.

It is a further aspect of at least one embodiment of this invention to provide for a delivery platform that will accommodate a unique patient positioning platform that allows patient positioning along a Y-axis (patient up and down), along a Z-axis (patient left/right), and rotational motion relative to the patent axis.

It is further aspect of at least one of the present embodiments to provide for a patient positioning system. The Patient Positioning System (PPS) will accurately position a human body for treatment with a radiosurgery system. Further, the PPS is also capable of accommodating and positioning other objects, such as animals, QA tools and cell cultures. The PPS provides mobility in 6-degrees-of-freedom to the Patient Positioning Table (PPT) with a movement envelope. With the use of active feedback from a camera system the PPS can provide positional accuracy on the order of ±0.1 mm. The PPS can also accommodate an on-board or detachable stereotactic system.

It is a further object of at least one aspect of this invention to provide a linear rail apparatus for supporting and positioning a patient in one axis, a rotary table in operative engagement with the linear rail system and capable of continuous movement with a linkage system wherein a table top of the rotary table assembly can be positioned within an operative two-dimensional plane.

It is a further object of at least one aspect of this invention to provide radiation beam delivery apparatus comprising:

at least one isotope-based treatment head:

at least six linear actuators, each actuator in independent operative engagement of the treatment head;

a support ring which secures the treatment head and each of the actuators, the support ring being supported by a support ring frame;

a drive motor in communication with the support ring, the drive motor able to bidirectionally rotate the support ring; and,

a brake on at least one side of the support ring for securing the support ring in a desired position.

It is a further object of at least one aspect of this invention to provide a a patient positioning system in operative engagement with a linear rail apparatus for supporting and positioning a patient in one axis having a rotary table in operative engagement with the linear rail system and capable of continuous movement with a linkage system wherein a table top of the rotary table assembly can be positioned within an operative two-dimensional plane of the radiation beam apparatus.

It is a further object of at least one aspect of this invention to provide a process of delivering a therapeutic dose of radiation to a human or animal patient comprising the steps of:

providing a radiation beam delivery apparatus having at least one isotope-based treatment head;

at least six linear actuators, each actuator in independent operative engagement of the treatment head;

a support ring which secures the treatment head and each of the actuators, the support ring being supported by a support ring frame;

a drive motor in communication with the support ring, the drive motor able to bidirectionally rotate the support ring;

a brake on at least one side of the support ring for securing the support ring in a desired position;

a patient positioning system in operative engagement with a linear rail apparatus for supporting and positioning a patient in one is having a rotory table in operative engagement with the linear rail system and capable of continuous movement with a linkage system wherein a table top of the rotary table assembly can be positioned within an operative two-dimensional plane of the radiation beam apparatus;

placing a patient on a platform;

moving the patient and the platform to a treatment position within the radiation beam delivery apparatus, the treatment position being controlled by the positioning of the treatment head and a collimator within the treatment head by the at least six actuators and further being controlled by positioning the patient by controlled movements of the table top along the patient's x-axis, the patients y-axis, rotational roll positioning along the patient's axis, and adjusting a pitch of the table top.

It is a further object of at least one aspect of this invention to provide a radiation beam delivery apparatus wherein a radiation shielding enclosure having a doorway is situated along a top surface and at least two sides of the beam delivery apparatus.

It is a further and more particular aspect of the invention to provide positioning system comprising:

a base member comprising a pair of rails, a platform supporting the rails along an upper platform surface, the platform responsive to a drive moter for moving the platform along the rails;

a rotating motorized platform supported on an upper surface of the platform;

a linkage system secured to the rotating motorized platform the linkage system providing vertical movement of a supported patient platform, the linkage system having a first, a second, a third and a fourth arm, each of the arms defining a pivot along a midpoint of the respective arms;

the first arm and the second arm attached along a respective lower end to a first side plate;

the third arm and fourth arm attached at a respective lower end to a second side plate;

a first rod connecting the lower end of the first arm, and the third arm;

a second rod connecting the lower end of the second arm and the fourth arm;

a third rod connecting the respective pivots of the first arm and the third arm;

a fourth rod connecting the respective pivots of the second arm and the fourth arm;

a fifth rod connecting an upper end of the first arm and the third arm;

a sixth arm connecting upper an upper end of the second arm and the fourth arm;

a first horizontal support member connecting the first arm and the second arm at a respective upper end of said arms, the first horizontal support member being further engaged by a first terminal end of the of the respective fifth and sixth rod;

a second horizontal support member connecting the third arm and the fourth arm at a respective upper end of said arms, the second horizontal support member being further engaged by a second terminal end of the of the respective fifth and sixth rod;

at least one drive motor for engaging at least one of the arm pivots, thereby raising and lowering in a coordinated manner the first and second horizontal support members; and,

a table assembly supported by the first and second support members the table assembly further comprising a horizontal table having a motor for directing the table along a patient axis, a pitching adjustment mechanism for compensating for deflection of the table by a patient's weight, the table assembly having independent movement in a horizontal 360 degree range of motion in response to movement of the rotating motorized platform.

These and other features, aspects, and advantages of the present invention will become better understood with reference to the following description and appended claims.

### BRIEF DESCRIPTION OF THE DRAWINGS

FIG. 1 is a perspective view of a beam delivery platform in conjunction with a patient positioning system.

FIG. 2 is a perspective view of a positioning system for positioning a treatment head, an imaging system, and a beam stopper supported by a ring gantry which in turn is supported by a roller support frame.

FIG. 3 is a perspective view components of drive frame and drive pedestal of the positioning system.

FIG. 4 i a side view of a treatment head Steward platform.

FIG. 5 is a bottom view of the treatment platform seen in FIG. 4.

FIG. 6 is a perspective view of a radiation shield that may be used with the beam delivery platform and associated components.

FIG. 7 is a perspective view of a patient positioning system.

FIG. 8 is a perspective view of a linear rail system used in a patent positioning system.

FIG. 9 is a parallel robotic system which connects the linear rail system to a table assembly of the patient positioning system.

FIG. 10 is a table assembly that is used with the patient positioning system.

A fully enabling disclosure of the present invention, including the best mode thereof to one of ordinary skill in the art, is set forth more particularly in the remainder of the specification, including reference to the accompanying drawings set forth in the specification.

### DESCRIPTION OF THE PREFERRED EMBODIMENT

Reference will now be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussionis is a description of exemplary embodiments only and is not intende as limiting the broader aspects of the present invention, which broader aspects are embodied in the exemplary constructions.

It is to be understood that the ranges mentioned herein include all ranges located within the prescribed range. As such, all ranges mentioned herein include all sub-ranges included in the mentioned ranges. For instance, a range from 100-200 also includes ranges from 110-150, 170-190, and 153-162. Further, all limits mentioned herein include all other limits included in the mentioned limits. For instance, a limit of up to 7 also includes a limit of up to 5, up to 3, and up to 4.5.

As used herein, the term “about” refers to a value plus or minus 10% of the stated value unless otherwise stated.

In describing the various figures herein, the same reference numbers used throughout to describe the same material, apparatus, or process pathway. To avoid redundancy, detailed descriptions of much of the apparatus once described in relation to a figure is not repeated in the descriptions of subsequent figures, although such apparatus or process is labeled with the same reference numbers.

One ability of the Beam Delivery Platform (BDP) is to precisely position radiation delivery heads for highly localized, conformal radiation dose distributions within clinical target volume with a sharp dose fall off at the boundary of the indication and the surrounding healthy tissue. The BDP can accommodate and hold single or multiple radiation therapy treatment heads including, isotope-based treatment heads like the PRTH, linear accelerator (LINAC) based treatment heads, and particle beam treatment heads. The BDP is capable of accurately positioning the selected irradiation focal spot generated by the on-board treatment head(s) system at a spatial isocenter with a positional accuracy on the order of ±0.1 mm. The BDP can deliver one or more isocenters along the Z axis of the center line of the ring 20, as well as to position the focal spot of the beam to any designated spatial point within the ring for non-isocentrical treatment. The BDP can move the treatment head(s) within an industry leading movement envelope that consists of 6-degrees-of-freedom (DOF). The BDP also features a radiation safety enclosure making it a self-contained system that doesn't require a radiation bunker.

The Beam Delivery Platform (BDP) allows precisely positioned radiation delivery heads for highly localized, conformal radiation dose distributions. The dosage delivered falls within clinical target volumes with a sharp dose fall off at the boundary of the indication and the surrounding healthy tissue. The BDP can accommodate and hold single or multiple radiation therapy treatment heads including, isotope-based treatment heads like the PRTH, linear accelerator (LINAC) based treatment heads, and particle beam treatment heads. The BDP is capable of accurately positioning the selected irradiation focal spot generated by the on-board treatment head(s) system at a spatial isocenter with a positional accuracy on the order of ±0.1 mm. The BDP can deliver one or more isocenters along the Z axis of the center line of the ring as well as to position the focal spot of the beam to any designated spatial point within the ring for non-iso-isocentrical treatment. The BDP can move the treatment head(s) within a movement envelope that consists of 6-degrees-of-freedom (DOF). The BDP also features an optional radiation safety enclosure making it a self-contained system that doesn't require a radiation bunker.

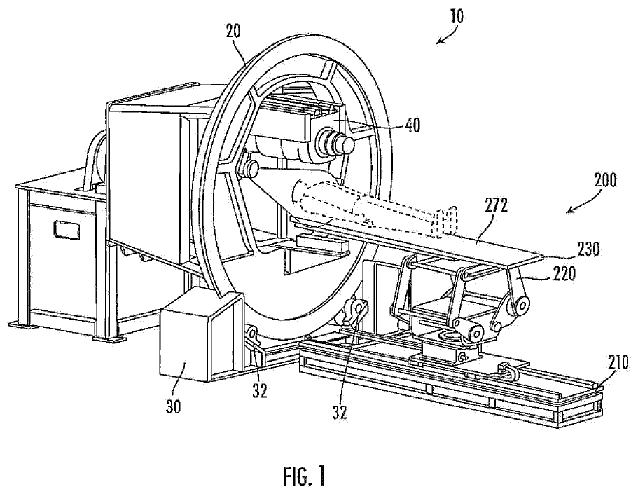

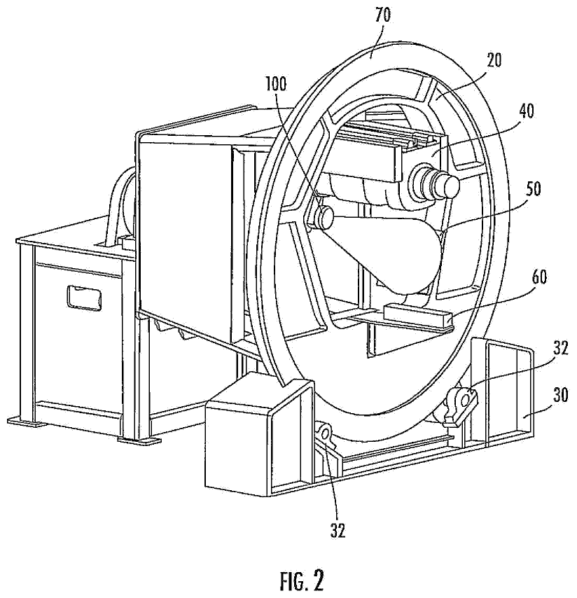

As best seen in reference to FIGS. 1-3, the BDP 10 has the following interconnected components including a ring 20 and a roller support frame 30 having rollers 32 that provides structural support and guides the rotational (motion to orbit the treatment head 40, imaging system 50 and beam stopper 60 around the patient. The ring gantry may be a solid structure or could be made from segmented pieces. The circular ring 20 may have various dimensions depending on the type of on-board radiation treatment head and/or the clinical or industrial application. The ring 20, as best seen in FIG. 5 may hold one or more on-board radiation treatment heads 40 via 6-12 DOF robotic platform(s) 80, a primary beam stopper system 60 and an on-board x-ray imaging system 100 that consists of one or more single of multiple focal spot x-ray tubes, collimators and flat panel detectors.

Both the x-ray sources and flat panel detectors can either be static and mounted directly to ring gantry 20, or mobile and mounted to ring 20 via a robotic platform. The x-ray sources and flat panel detectors can be mounted in-plane with or at an oblique angle to the ring gantry, and they can provide fluoroscopy, stereo-imaging, tomosynthesis and cone beam CT imaging for pre-treatment imaging and image guidance during treatment. The ring 20 can also support and spatially accommodate other imaging modalities like MRI, CT, PET, Ultrasound and SPECT. A brake ring 70 is mounted on the front face of the ring gantry.

The roller support frame 30 provides structural support for the ring 20. Two or more rollers 32 are mounted on the base of the roller support frame 30 to guide the rotational motion of the ring gantry about the z-axis and to prevent axial shifts of the ring gantry along the z-axis. When the ring 20 is driven the outer surface will roll on the supporting surface of the rollers. The roller support frame also houses two brakes, one on either each side of the roller support frame and which engage the brake ring 70.

The drive frame 90 is attached to back face of the ring 20. The main function of the drive frame 90 is to transfer the power from the drive motor 92 to the ring gantry during rotation about the z-axis. The drive frame depicted has a rectangular pyramid shape, however, the shape and dimensions of the drive frame 92 are variable depending on the application.

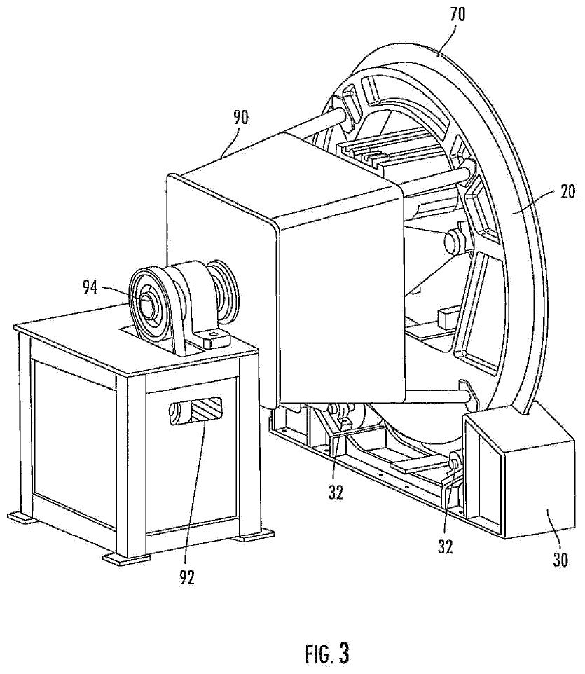

A drive shaft 94 is mounted to the back face of the drive frame and the top face of the drive pedestal and may use with either a bearing and bearing housing coupling 96 or a bushing and bushing housing (not shown). The drive shaft 94 provides a housing for a slip ring, which enables cable management for unlimited, bi-directional continuous rotation of the ring gantry about the z-axis. The main function of the drive pedestal 100 is to provide support to the drive frame 90 and house the drive train components. The drive pedestal houses the drive motor 92 and a gearbox that communicates with the drive motor. The motor and gear gearbox allow for continuous bi-directional rotation of the ring gantry about the z-axis. The drive motor and gearbox attach to the drive shaft 94 either by a pulley and gear system, as seen in FIG. 3, rack and pinon, or directly to the shaft depending on the application.

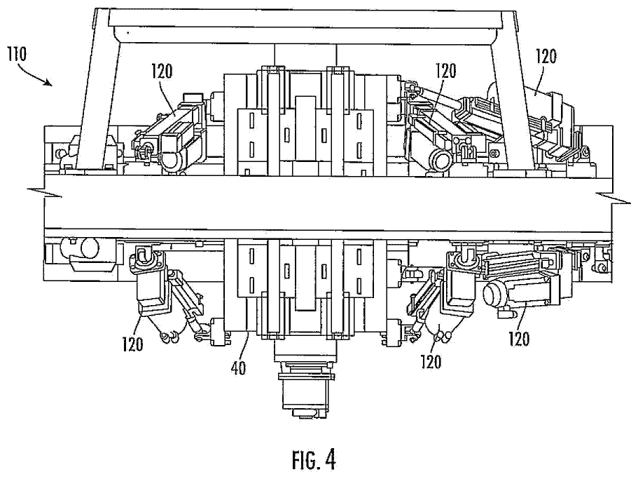

The treatment head having collimators therein is positioned on a Steward platform 110 seen in FIGS. 4 and 5 and provides 6 independent linear actuators 120 that move the treatment head(s) 40 with 6 DOF (x, y, z, roll, pitch, and yaw) with ±0.1 mm accuracy. The treatment head Steward platform 110 serves two purposes: 1) to move the treatment head(s)'s focal spot to the spatial isocenter, to accommodate treatment heads with multiple focal spot features: 2) to move the treatment head(s) to any spatial point outside of the spatial isocenter to perform advanced dose delivery techniques such as dose painting, etc.

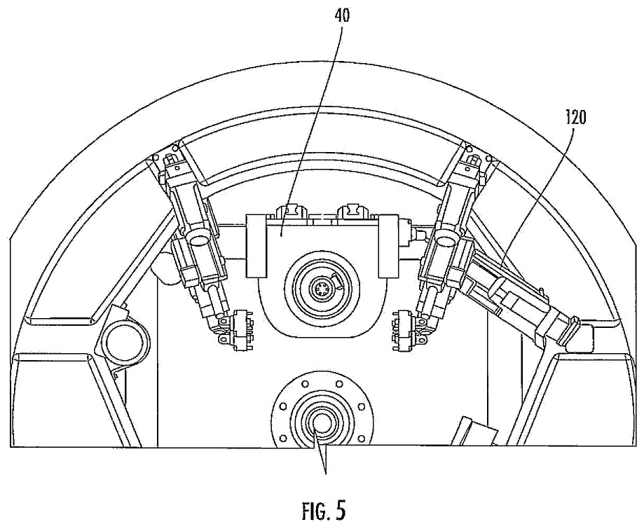

The radiation shielding enclosure 120 seen in FIG. 6 encompasses the entire treatment area including the BDP and PPT. It may have a door 122, shown partially raised, which may slide or otherwise open, either manually or motor driven, to allow access to the interior of the shielding enclosure 120. The enclosure as seen has a tunnel-like shape and should enclose the top and three sides though the final dimensions and shape may be modified to reflect the end application components. Since the BDP incorporates a primary beam stopper system, the function of the radiation shielding enclosure 120 is to provide enough shielding for scattered radiation to make the system self-contained (i.e., no further radiation shielding—such as a bunker—required).

In isocenter based treatment systems, the positional accuracy of the isocenter is crucial. To establish a high degree of accuracy, the treatment head Steward platform will allow movement of the treatment head with 6 DOF and with ±0.1 min positional accuracy. This Steward Platform 110 allows the BDP system to correct for isocenter positional deviations due to mechanical and machining inaccuracies, changes due to normal wear and tear caused by operation overtime and inaccuracies due to collimator focusing. Conventional isocenter—based treatment heads do not utilize a treatment head Steward platform or similar system to finely adjust the position of the isocenter. The system described herein will maintain an isocenter position that will not vary around the desired spatial isocenter, thereby avoiding the negative impacts of a radiation dose fall off and resulting damage to healthy tissue.

In some embodiments of the present invention, the BDP may be utilized with a patient positioning system (PPS) 200 which will accurately position a human body, an animal, cell cultures, tools or other items for use with the BDP based radiosurgery system. With the use of active feedback from a camera system the PPS can provide positional accuracy on the order of ±0.1 mm. The PPS can also accommodate an on-board or detachable stereotactic system and alloww for real time adjustments for dosage patterns and interval for moving tumors.

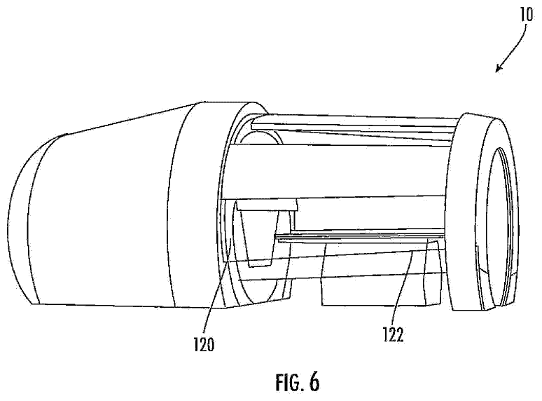

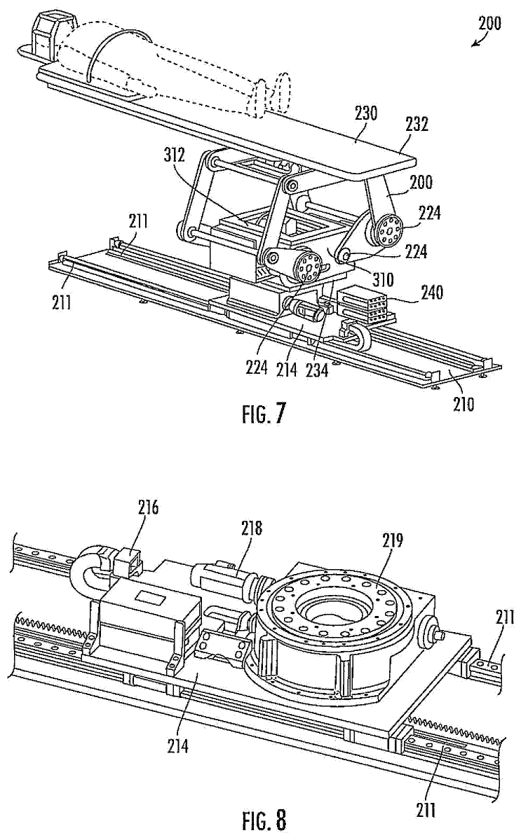

The PPS 200 seen in FIG. 7 is comprised of three interconnected components which include the linear rail system 210, a parallel robot system 220, and a table assembly 230.

The linear rail system 210 seen in FIG. 8 is responsible for moving the components of the positioning system in one axis by moving a support plate 234 relative to the bottom base plate 214 of the rail system 210. A rotary table 232 is attached to the support plate 234 and it can rotate continuously via the linkage system of the parallel robot system 220 (FIG. 9) which in turn is rotated by the movement of the rotating platform 219 driven by motor 218. Using the linear motion supplied by motor 224, the patient and table 232 can be pushed in and pulled out of the operating area along rails 211 of rail system 210. Using the rotary table 230, the patient can be rotated a 360° range of motion so the patient can be inserted into an in-room imagining system (CT, MR, PET, DSA, etc.) if the operating room is equipped with one. For both movements, the respective motors are equipped with a primary and secondary encoder to ensure the precision required for treatment. The linear rail system 210 provides a connection point 216 for communication cables which link the system to the external controllers and processors.

The parallel robot system 220 seen in FIG. 9 has four pairs of parallel arms 222 that are linked with high precision shafts 221 that ensure the parallel arms 222 can accurately position the patient. Three sets of servo motors and gearboxes 224 are used to drive three joints 225 of arms 222 to position the PPS table 232 anywhere in a 2D plane. All three motors 224 are redundantly encoded to ensure the precision required for the fine motor movements when adjusting the patient's position to correct for target motions. Due to the high mobility of this part, the table can lower to a comfortable level for patient setup and can elevate to the height required for treatment. The Parallel Robot System has 3 DOF, namely: motion along the Y-axis (patient up/down), along the X-axis (patient left/right) and patient rotational motion (Roll) along the patient axis. The linkage attaches to a base plate 212 that is mounted to the rotary table and a top plate 236 that attaches to the table assembly described below.

The parallel robot system 220 further comprises a base member having a pair of rails, a platform supporting the rails 211 along an upper platform surface, the platform 234 responsive to a drive motor 240 for moving the platform along the rails 211. A rotating motorized platform 219 is supported above an upper surface of the rail platform 214 and a linkage system 220 secured to the rotating motorized platform 219 provides vertical movement of a supported patient platform 234, the linkage system having a first 301, a second 302, a third 303 and a fourth 304 arm, each of the arms 301-304 defining a pivot along a midpoint of the respective arms. The first arm and the second arm are attached along a respective lower end to a first side plate 310 and the third arm and the fourth arm are attached at a respective lower end to a second side plate 312. A first rod 320 connects the lower end of the first arm and the third arm and a second rod 322 connects the lower end of the second arm and the fourth arm. A third rod 324 connects the respective pivots of the first arm and the third and a fourth rod 326 connects the respective pivots of the second arm and the fourth arm. A fifth rod 328 connects an upper end of the first arm and the upper end of the third arras while a sixth arm 330 connects an upper end of the second arm and the fourth arm.

A first horizontal support member 340 connects the first arm and the second arm at a respective upper end of the arms, the first horizontal support member being further engaged by a first terminal end of the of the respective fifth and sixth rod. A second horizontal support member 342 connects the third arm and the fourth arm at a respective upper end of the arms, the second horizontal support member being further engaged by a second terminal end of the of the respective fifth and sixth rod.

There is at least one drive motor 224 for engaging at least one of the arm pivots, thereby raising and lowering in a coordinated manner the first and second horizontal support members. As illustrated, three drive motors 224 can be used.

The table assembly 220 is supported by the first and second support members and the table assembly further comprises a horizontal table 232 having a motor 240 for directing the table 232 along a patient axis, a pitching adjustment mechanism 238 for compensating for deflection of the table 232 by a patient's weight, the table assembly having independent movement in a horizontal 360 degree range of motion in response to movement of the rotating motorized platform.

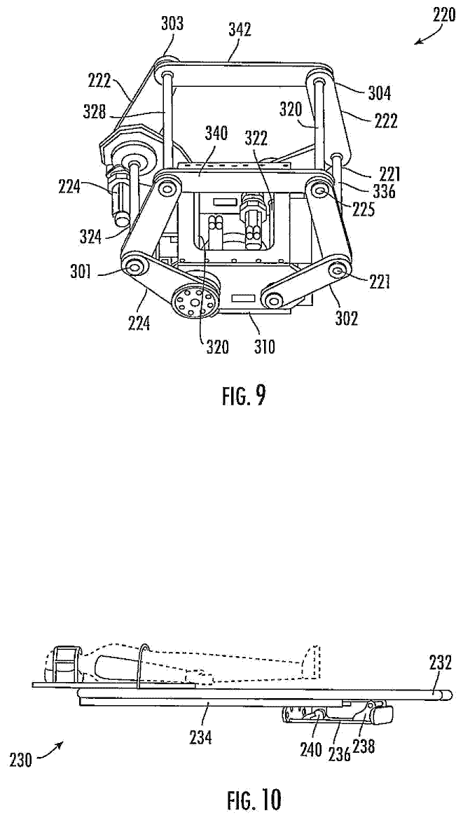

The table assembly 230 seen in FIG. 10 is the upper part the PPS system, The assembly connects via top plate 236 to the parallel robot apparatus 220. A motor 240 is used to move the tabletop 232 in a x-axis patient direction. The tabletop 232 is a radiation transparent (e.g. carbon fiber) outer shell filled with resin, to minimize the absorption of the treatment and imaging radiation beams. Since the tabletop is cantilevered, it will experience a small amount of deflection that varies with the weight of the patient. Therefore, a pitching mechanism 238 is utilized with the table assembly to couteract this deflection and keep the operating area of the system level. Also, the pitching mechanism has an angular range of ±3 degrees to accommodate patient positioning requirements.

Various radiosurgery techniques are known in the art as reflected in U.S. Pat. Nos. 7,318,805 and 5,207,223, and 5,769,861. These three patents are incorporated herein by reference. Additionally, the use of applicable software and control hardware mechanisms that are utilized in prior art radiosurgery apparatuses and processes are well know to one of ordinary skill in the art. Such apparatus and techniques can be used with the present apparatuses and processes described herein.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention. In addition, it should be understood that aspects of the various embodiments may be interchanged, both in whole, or in part. Therefore, the spirit, and scope of the invention should not be limited to the description of the preferred versions contained therein.

That which is claimed:

1. A patient positioning system comprising:
a linear rail apparatus for supporting and positioning a patient in one axis, a rotary table in operative engagement with the linear rail system and capable of continuous movement with a linkage system wherein a table top of the rotary table assembly can be positioned within an operative two-dimensional plane; and a pitching adjustment mechanism for compensating for deflection of the table by a patient's weight.

2. A radiation beam delivery apparatus comprising:
at least one isotope-based treatment head;
at least six linear actuators, each actuator in independent operative engagement of the treatment head;
a support ring which secures the treatment head and each of the actuators, the support ring being supported by a support ring frame;
a drive motor in communication with the support ring, the drive motor able to bidirectionally rotate the support ring; and,
a brake on at least one side of the support ring for securing the support ring in a desired position.

3. The radiation beam delivery apparatus according to claim 2 wherein the system further comprises a patient positioning system in operative engagement with a linear rail apparatus for supporting and positioning a patient in one axis having a rotary table in operative engagement with the linear rail system and capable of continuous movement with a linkage system wherein a table top of the rotary table assembly can be positioned within an operative two-dimensional plane of the radiation beam apparatus.

4. A process of delivering a therapeutic dose of radiation to a human or animal patient comprising the steps of:
providing a radiation beam delivery apparatus having at least one isotope-based treatment head;
at least six linear actuators, each actuator in independent operative engagement of the treatment head;
a support ring which secures the treatment head and each of the actuators, the support ring being supported by a support ring frame;
a drive motor in communication with the support ring, the drive motor able to bidirectionally rotate the support ring;
a brake on at least one side of the support ring for securing the support ring in a desired position;
a patient positioning system in operative engagement with a linear rail apparatus for supporting and positioning a patient in one axis having a rotary table in operative engagement with the linear rail system and capable of continuous movement with a linkage system wherein a table top of the rotary table assembly can be positioned within an operative two-dimensional plane of the radiation beam apparatus;
placing a patient on a platform;
moving the patient and the platform to a treatment position within the radiation beam delivery apparatus, the treatment position being controlled by the positioning of the treatment head and a collimator within the treatment head by the at least six actuators and further being controlled by positioning the patient by controlled movements of the table top along the patient's x-axis, the patient's y-axis, rotational roll positioning along the patient's axis, and adjusting a pitch of the table top.

5. A radiation beam delivery apparatus according to claim 1 wherein a radiation shielding enclosure having a doorway is situated along a top surface and at least three sides of the beam delivery apparatus.

6. A patient positioning system comprising:
a base member comprising a pair of rails, a platform supporting the rails along an upper platform surface, the platform responsive to a drive motor for moving the platform along the rails;
a rotating motorized platform supported on an upper surface of the platform;
a linkage system secured to the rotating motorized platform the linkage system providing vertical movement of a supported patient platform, the linkage system having a first, a second, a third and a fourth arm, each of the arms defining a pivot along a midpoint of the respective arms;
the first arm and the second arm attached along a respective lower end to a first side plate;
the third arm and the fourth arm attached at a respective lower end to a second side plate:
a first rod connecting the lower end of the first arm and the third arm;
a second rod connecting the lower end of the second arm and the fourth arm;
a third rod connecting the respective pivots of the first arm and the third arm;
a fourth rod connecting the respective pivots of the second arm and the fourth arm;
a fifth rod connecting an upper end of the first arm and the third arm;
a sixth arm connecting an upper end of the second arm and the fourth arm;
a first horizontal support member connecting the first arm and the second arm at a respective upper end of said arms, the first horizontal support member being further engaged by a first terminal end of the of the respective fifth and sixth rod;
a second horizontal support member connecting the third arm and the fourth arm at a respective upper end of said arms, the second horizontal support member being further engaged by a second terminal end of the of the respective fifth and sixth rod;
at least one drive motor for engaging at least one of the arm pivots, thereby raising and lowering in a coordinated manner the first and second horizontal support members; and,
a table assembly supported by the first and second support members the table assembly further comprising a horizontal table having a motor for directing the table along a patient axis, a pitching adjustment mechanism for compensating for deflection of the table by a patient's weight, the table assembly having independent movement in a horizontal 360 degree range of motion in response to movement of the rotating motorized platform.

7. The radiation beam delivery apparatus according to claim 2 wherein the treatment head can apply targeted radiation along one or more isocenters along the Z axis of the center line of the ring.

8. The radiation beam delivery apparatus according to claim 2 wherein the treatment head can apply a targeted radiation beam to any designated spatial point within the ring for non-isocentrical treatment.

9. The radiation beam delivery apparatus according to claim 2 wherein the ring can additionally support other imaging modalities selected from the group consisting of MRI, CT, PET, ultrasound, and Spect.

10. The radiation beam delivery apparatus according to claim 2 wherein the ring further defies a brake ring mounted on a face of the ring and responsive to a braking pressure supplied by at least one braking member.

11. The radiation beam delivery apparatus according to claim 2 wherein the operation of the six actuators allows for isocentrical positioning of a target beam and which compensates for positional deviations that may otherwise occur from mechanical wear, machining inaccuracies, and variations in collimator focal points.

12. A patient positioning system according to claim 6 wherein a stereotactic camera system is present that interacts in real time to monitor at least on of a dosage pattern or an interval period for irradiation of moving tumors.

13. A patient positioning system according to claim 6 wherein the 360-degree range of motion facilitates the positioning of the platform surface from a first position within a beam delivery apparatus to a second position within a separate, stand-alone imaging system.

14. A patient positioning apparatus according to claim 1 wherein a stereotactic system is present that allows for real time dosage pattern modifications and timing intervals for radiation delivery for a moving tumor.

15. A patient positioning apparatus according to claim 2 wherein the a steward head is used to mount the six linear actuators and the treatment head, thereby allowing signals from a stereotactic system to be altered in real time used to vary in real a dosage pattern modifications and a timing interval for radiation delivery for a moving tumor.

16. A patient positioning apparatus according to claim 2 wherein an imaging system is present which allows x-ray sources and flat panel detectors to be mounted in iso-centrical or non-isocentrical positions to provide for pre-treatment imaging and image guidance with fluoroscopy, stereo-imaging, tomosynthesis and cone beam CT imaging.
